(19) United States
(12) Patent Application Publication
(10) Pub. No.: US 2009/0110663 A1
(43) Pub. Date: Apr. 30, 2009

(54) Method for treatment of bowel disorders

(76) Inventor: George M. Halow, El Paso, TX (US)

Correspondence Address: David G. Moore;Strasburger & Price, LLP, Suite 4400, 901 Main St., Dallas, TX, 75202, US

(21) Appl. No.: 11/977,221

(22) Filed: Oct. 24, 2007

(51) Int. Cl. A61K 35/66; A61K 35/74; A61P 1/00

(52) U.S. Cl. 424/93.3; 424/93.1; 424/93.45; 424/93.51

(57) ABSTRACT

A composition and method for treatment for bowel disorders is described. The composition consists of two parts. First, highly purified, insoluble, chemically unmodified fiber from plants is mixed with an aqueous solution such as water. The second component of the composition is a commercially available probiotic preparation. These commercially probiotic preparations contain large numbers of viable microorganisms of either Lactobacillus sp., Bifidobacter sp. Streptococcus sp. or yeast. Consumption of the fiber preparation or probiotic by patients with constipation secondary to surgery or chronic antibiotic therapy, ulcerative colitis or Crohn's disease results in either a resolution of the symptoms or markedly improvement in symptoms without significant side effects.

### CROSS-REFERENCE TO RELATED APPLICATIONS

None.

### STATEMENT REGARDING FEDERALLY SPONSORED RESEARCH OR DEVELOPMENT

The invention described in this application was not the subject of federally sponsored research or development.

### BACKGROUND

1. Field

The field of the invention disclosed in this application is a method of medical or veterinary treatment. Specifically, a method employing probiotics in combination with fiber for the treatment of intestinal disorders such as ulcerative colitis, constipation secondary to surgery or chronic use of antibiotics, Crohn's disease and maintenance of general bowel health is disclosed.

2. Description of the Related Art

The alimentary tract of most mammals consists of a long tube through the body. The alimentary tract begins at the mouth which is attached to the stomach by the esophagus. The stomach in turn in connected to the small intestine which is subsequently attached to the large intestine or colon. The colon ends at the rectum. Food enters the alimentary tract through the mouth and travels to the stomach where acids and various enzymes break down the food. Nutrients are absorbed in the small intestine and water is removed from the digested food by the colon. The colon also packages the digested food into mostly solid parcels for exit through the rectum. In the intestine there exists enormous numbers of bacteria of many varieties. Because of the large diversity of organisms, no single organism is permitted to become dominant. In this way the intestine protects itself from invasion by pathogenic microorganisms.

The microorganisms which reside in the intestine also provide beneficial effects for the host organism. These beneficial effects include breaking down ingested food into component parts for ease of assimilation by the host. The bacteria also use the food ingested by the host to produce valuable nutrients such as vitamins. When this delicate balance of diversity of microorganisms is disturbed, the effects may be transferred to the host in the form of sickness and disease.

Probiotics are mixtures of viable microorganisms, mainly bacteria which commonly reside in the intestine, and which have been packaged in such a manner so as to be able to be easily ingested by a mammalian host. Probiotics have been used to restore the diversity of the microbial ecology in the intestine by ingestion of preparations containing various types of viable bacteria. It is hoped that this ingestion of the probiotic preparations will restore the diversity of microbes present in the intestine and promote intestinal health and well-being.

Probiotics have also been used to treat specific intestine-related diseases. In a patent to Neeser et al. (U.S. Pat. No. 6,835,376) a probiotic preparation of Lactobacillus paracasei was used to treat diarrhea by preventing the pathogenic bacteria from attaching to the cells lining the intestine, a prerequisite for the development of diarrhea caused by many bacteria.

In a patent issued to Perry (U.S. Pat. No. 6,203,797) a dietary supplement was prepared which, optionally, could contain probiotic preparations of “friendly” bacteria such as Lactobacillus bulgaricus, Lactobacillus acidophilus, Lactobacillus plantarum and Bifidobacterium bifidum.

A probiotic preparation of Enterococcus faecium, was used to treat irritable bowel syndrome in a patent issued to Alleb et al. (U.S. Pat. No. 5,589,168)

Uremia related to kidney failure was treated with probiotics consisting of ammoniaphilic bacteria, and sorbents to remove toxins in a patent issued to Ranganathan et al. (U.S. Pat. No. 6,706,287)

### BRIEF SUMMARY OF THE INVENTION

The embodiments of the invention disclosed herein relate to the treatment of certain bowel disorders with a mixture of highly purified fiber and various probiotic preparations. Conditions such as constipation following antibiotic therapy, or secondary to surgery are efficiently relieved by administration of a solution of highly purified fiber and probiotic preparations. Other intestinal disorders such as Crohn's disease and ulcerative colitis also respond favorably following administration of probiotic with a solution of fiber. Both the fiber preparation and the probiotic preparations are commercially available enabling this novel treatment to be administered quickly, easily and economically effectively.

### BRIEF DESCRIPTION OF THE SEVERAL VIEWS OF THE DRAWINGS

None.

### DETAILED DESCRIPTION OF THE INVENTION

Disorders of bowel function can be manifested by protean symptoms. Pain, bloating, gas, diarrhea, constipation and bleeding, among others, can all be manifestations of bowel disorder. Some bowel disorders are iatrogenic. Constipation can result from prolonged antibiotic therapy or occur after surgery. Other bowel conditions can be chronic in duration. Ulcerative colitis is an inflammatory disease of the bowel which results in the formation of discrete ulcers in the tissues of the intestines. Crohn's disease is also an inflammatory disease of the bowel in which the tissues lining the lumen of the intestine and colon may be inflamed.

Many types of treatment have be tried in order to alleviate suffering caused by bowel disorders. Most of these treatments have been unsuccessful or only partially successful at alleviating the discomfort caused by these conditions. The invention disclosed herein is a method and composition for the treatment of the aforementioned bowel illnesses. The method and composition disclosed herein employs the administration of fiber in conjunction with probiotics to individuals suffering from bowel disorders. The fiber is highly-purified, chemically unmodified and suspended in an aqueous solution such as water or fruit juice to improve palatability. The fiber is plant-derived cellulose which is insoluble or only partially soluble in aqueous solutions. Approximately 10-25 grams of the aforementioned fiber are suspended in a large glass of water, for example and consumed one time per day. The quantity of water needed is at a minimum in the range of 30-60 mls.

In other embodiments of the invention disclosed herein commercially—available sources of fiber may be used in the amounts described on the package label. Those other sources of fiber which are described as follows:

1. Algin (Greatest Herbs on Earth) is a nondigestible fiber derived from kelp or brown seaweed. Two capsules taken three times daily with two glasses of juice is the standard dosage. Each capsule contains 475 mg. of sodium alginate.
2. Benefiber (Novartis Consumer Health, Inc.) is a nonbulking fiber made from wheat dextrin. Benefiber is gluten free and taken at the rate of 2 teaspoons of Benefiber suspended in 4-8 ozs of aqueous media three times a day.
3. Fibersure (Procter & Gamble) is a fiber product composed of insulin. The standard dosage is 5 grams fibersure in one glass of water or other aqueous fluid.
4. Metamucil (Proctor & Gamble) is a fiber product made from Psyllium husk and ingested at the rate of one teaspoon in 8 ozs of aqueous media, three times per day.

The probiotic is commercially available and is taken according to manufacturer's instructions. The commercially available probiotics employed herein include the following:

1. CP-1 (Custom Probiotics, Inc.) CP-1 consists of Lactobacillus acidophilus, Lactobacillus plantarum, Lactobacillus rhamnosus, Bifidobacterium bifidum and Bifidobacterium longum. Information provided by the manufacturer indicates that 2 capsules of CP-1 should be taken per day. Each capsule contains 35 billion microorganisms.
2. Culturelle (Amerifit Nutrition, Inc.) Culturelle is composed of Lactobacillus GG with 10 billion viable bacteria per capsule. One-two capsules per day constitute the ordinary dosage of Culturelle.
3. Flora Q (Kenwood Therapeutics) Flora Q is composed of Bifidobacterium, Lactobacillus acidophilus, Lactobaccillus paracasei and Streptococcus thermophilus. One capsule of Flora Q containing no less than 4 million CFU/ml (colony forming units/milliliter) is the standard daily dosage.
4. Florastor (Bicodex, Inc.) Florastor is composed of lyophilized Saccharomyces, boulardii, a yeast. One capsule, containing 5 billion live cells per capsule, taken twice daily is the standard dosage.
5. HLC High Potency capsules (Emerson Ecologics, Inc.) HLC High Potency capsules are composed of two different strains of Lactobacillus acidophilus, and one strain each of Bifidobacterium bifidum and Bifidobacterium lactis. Each capsule contains 8 billion viable bacteria. Two capsules per day taken with meals is the normal dosage.
6. Lacidofil DF (Xymogen, Inc.) Lacidofil DF consists of Bifidobacterium longum, Lactobacillus acidophilus and Lactobacillus rhamnosus. Each capsule contains 2 billion live organisms. One to two capsules per day are taken before or during meals.
7. Ultra Flora plus DF (Metagenics) Ultra Flora plus DF is composed of Bifidobacterium lactis and Lactobacillus acidophilus. Each capsule contains no less than 5 billion live organisms. The normal dosage is 1-2 capsules per day.
8. VSL#3 (Sigma-Tau Pharmaceuticals, Inc.) VSL#3 consists of Bifidobacterium breve, Bifidobacterium infantis, Bifidobacterium longum, Lactobacillus acidophilus, Lactobacillus bulgaricus, Lactobacillus casei, Lactobacillus plantarum, and Streptococcus thermophilus. Each packet of VSL#3 contains 450 billion live lactic acid bacteria. One/half to 8 packets per day is the normal dosage.

Approximately 10-25 grams of highly purified fiber which is plant derived, chemically unmodified and insoluble or partially insoluble in aqueous media such as water is suspended in a minimum of 30-60 mls of the aqueous media. Fruit juice may be substituted for water to improve palatability of the fiber. This quantity of fiber is consumed one time per day. In addition, one of the commercially available probiotics is also ingested. The daily dosage of probiotic to be administered with the fiber is either 2 capsules of CP-1, 1-2 capsules of Culturelle, Flora Q (1 capsule), Florastor (1 capsule, twice per day), HLC (2 capsules with meals), Lacidofil (1-2 capsules prior to or with meals), Ultraflora (1-2 capsules) or VSC#3 (0.5-8 packets. Alternatively, a commercially-available form of fiber ingested in the amounts described by the manufacturer may be substituted for the aforementioned fiber preparation. The above described therapeutic combination incorporating fiber and probiotic are used in each of the embodiments of the disclosed invention as described below.

### FIRST EMBODIMENT

Patients undergoing surgery frequently have narcotics and other medicines administered prior to, and during surgery to relieve pain and relax muscles. A common side effect of these medicines, especially the narcotic medicines is to decrease motility of the intestine resulting in constipation. This constipation following surgery is usually self-limiting. However, in some circumstances the constipation can be severe, long-lasting and result in fecal impactions and intestinal obstruction requiring medical/surgical intervention. Fiber has been used as a treatment for constipation. However, in this disclosure, fiber in conjunction with commercially-available probiotics is administered to patients with post-surgical constipation. Patients with post-surgical constipation who receive the aforementioned administration of fiber and probiotic are relieved of the constipation quicker than with just fiber or probiotic alone. Further, these post surgical patients who are administered fiber plus probiotic recover from their constipation with few, if any side effects.

### SECOND EMBODIMENT

Patients undergoing long-term or chronic antibiotic therapy frequently develop constipation presumably because of the disruption in the diversity of the microbial flora of the intestine caused by the administration of the antibiotic. Fiber has been used alone to treat the symptoms of constipation in patients with antibiotic associated constipation. However, fiber plus probiotic not only relieves the symptoms but also helps quickly restore the normal balance of microbial diversity in the intestines. Patients receiving both fiber and probiotics have fewer relapses of constipation than those individuals receiving fiber alone.

### THIRD EMBODIMENT

Crohn's disease is a chronic disorder of the gastrointestinal tract characterized by inflammation. It is unclear as to the cause of Crohn's disease but it may be an auto-immune disease in which the body mistakenly recognizes the body as foreign and attacks it with inflammation as the result. The inflammation, if it persists for a sufficient period of time can result in a blockage of the intestine, a medical emergency. There is no cure and many treatments have been used in an attempt to ameliorate the symptoms of Crohn's disease. Patients suffering from the symptoms of Crohn's disease who receive fiber plus probiotic have milder symptoms and shorter periods of time in which symptoms are present as compared to other forms of treatment.

### FOURTH EMBODIMENT

Ulcerative colitis is a disease of the gastrointestinal tract which is manifested by inflammation and the development of ulcers in the tissues lining the gastrointestinal tract. There is no cure for ulcerative colitis and the goal of therapy is to moderate the symptoms. Patients administered fiber plus probiotic have fewer and more moderate symptoms which last a shorter period of time than do those patients receiving alternate therapies.

There are other bowel disorders and other combination of fiber and probiotic not specifically disclosed herein. A person of ordinary skill in the art would recognize that such combination exist. Those combinations even that not specifically disclosed herein are included within the scope and meaning of the claims appended hereto.

I claim:

1. a combination therapy for gastrointestinal disorders in mammals comprising
aqueous media containing fiber; and
a probiotic.

2. the combination therapy defined in claim 1 wherein said fiber is chemically unmodified.

3. the combination therapy defined in claim 1 wherein said fiber is insoluble or partially insoluble in aqueous media.

4. the combination therapy defined in claim 1 wherein said fiber is plant derived cellulose.

5. the combination therapy defined in claim 1 wherein approximately 10-25 grams of said fiber is dissolved in at least 30-60 mls of aqueous media.

6. the combination therapy defined in claim 1 wherein said probiotic is CP-1 taken in a dosage of about two capsules per day.

7. the combination therapy defined in claim 1 wherein said probiotic is Culturelle taken in a dosage of about 1-2 capsules per day.

8. the combination therapy defined in claim 1 wherein said probiotic is Flora Q taken in a dosage of about one capsule per day.

9. the combination therapy defined in claim 1 wherein said probiotic is Florastor taken in a dosage of about two capsules per day.

10. the combination therapy defined in claim 1 wherein said probiotic is HLC taken in a dosage of two capsules per day.

11. the combination therapy defined in claim 1 wherein said probiotic is Lacidofil taken in a dosage of about one to two capsules per day.

12. the combination therapy defined in claim 1 wherein said probiotic is Ultraflora plus DF taken in a dosage of about 1-2 capsules per day.

13. the combination therapy defined in claim 1 wherein said probiotic is VS. #3 taken in a dosage of about one half to eight packets per day.

14. the combination therapy defined in claim 1 wherein said combination therapy is administered to mammals with constipation secondary to surgery.

15. the combination therapy defined in claim 1 wherein said combination therapy is administered to mammals with constipation secondary to chronic antibiotic therapy.

16. the combination therapy defined in claim 1 wherein said combination is administered to mammals with Crohn's disease.

17. the combination therapy defined in claim 1 wherein said combination is administered to mammals with ulcerative colitis.

18. the combination therapy defined in claim 1 wherein said mammal is a human.

19. A method for treating mammals with gastrointestinal disorders including:
mixing fiber in an aqueous media; and
administering a probiotic and said fiber to mammals with gastrointestinal disorders.

20. The method for treating mammals with gastrointestinal disorders as defined in claim 19 wherein said fiber is unmodified.

21. The method for treating mammals with gastrointestinal disorders as defined in claim 19 wherein said fiber is insoluble or partially soluble in aqueous media.

22. The method for treating a mammal with gastrointestinal disorders as defined in claim 19 wherein said fiber is plant derived cellulose.

23. The method for treating a mammal with gastrointestinal disorders as defined in claim 19 wherein 10-15 grams of said fiber is suspended in at least 30-60 mls of aqueous media.

24. The method for treating a mammal with gastrointestinal disorders as defined in claim 19 wherein said probiotic is selected from the group consisting of CP-1, Culturelle, Flora Q, Florastor, HLC, Lacidofil, Ultraflora, and VSC#3.

25. The method for treating a mammal with gastrointestinal disorders as defined in claim 19 wherein said method is administered to mammals with constipation secondary to surgery.

26. The method for treating a mammal with gastrointestinal disorders as defined in claim 19 wherein said method is administered to mammals with constipation secondary to chronic antibiotic therapy.

27. The method for treating a mammal with gastrointestinal disorders as defined in claim 19 wherein said method is administered to mammals with Crohn's disease.

28. The method for treating a mammal with gastrointestinal disorders as defined in claim 19 wherein said method is administered to mammals with ulcerative colitis.

29. the method defined in claim 19 wherein said probiotic is CP-1 taken in a dosage of about two capsules per day.

30. the method defined in claim 19 wherein said probiotic is Culturelle taken in a dosage of about 1-2 capsules per day.

31. the method defined in claim 19 wherein said probiotic is Flora Q taken in a dosage of about one capsule per day.

32. the method defined in claim 19 wherein said probiotic is Florastor taken in a dosage of about two capsules per day.

33. the method defined in claim 19 wherein said probiotic is HLC taken in a dosage of two capsules per day.

34. the method defined in claim 19 wherein said probiotic is Lacidofil taken in a dosage of about one to two capsules per day.

35. the method defined in claim 19 wherein said probiotic is ultraflora plus DF taken in a dosage of about 1-2 capsules per day.

36. the method defined in claim 19 wherein said probiotic is VSC #3 taken in a dosage of about one half to eight packets per day.

37. the method defined in claim 19 wherein said mammal is a human.
